(19) United States
(12) Patent Application Publication
(10) Pub. No.: US 2022/0051789 A1
(43) Pub. Date: Feb. 17, 2022

(54) DETERMINING INTERRUPTIBILITY BY TRACKING A USER'S PROGRESS

(71) Applicant: INTERNATIONAL BUSINESS MACHINES CORPORATION, Armonk, NY (US)

(72) Inventors: James G. Thompson, Escondido, CA (US); Sun Young Park, San Diego, CA (US); Dustin Michael Sargent, San Diego, CA (US)

(21) Appl. No.: 16/947,669

(22) Filed: Aug. 12, 2020

(51) Int. Cl. G16H 40/60; G06N 20/00; G16H 30/20

(52) U.S. Cl. CPC G16H 40/60; G16H 30/20; G06N 20/00

(57) ABSTRACT

A method, computer system, and a computer program product for determining interruptibility is provided. The present invention may include gathering data about a task performed by a user. The present invention may include training a machine learning model based on the gathered data. The present invention may include determining a task estimate. The present invention may include tracking a task performance of the user in real time. The present invention may include determining an interruptibility of the user. The present invention may include providing the interruptibility of the user.

### BACKGROUND

The present invention relates generally to the field of computing, and more particularly to progress tracking systems.

Imaging may play an important role in modern medicine. Imaging techniques, including, but not limited to, X-rays, ultrasounds, CT scans, and MRIs, may depict details of a patient's body. Digital Imaging and Communications in Medicine (“DICOM”) may be a standard format that enables medical professionals to view, store, and share medical images irrespective of their geographic location and/or the devices they use, as long as those devices support the DICOM format. The images, along with the corresponding patient data, may be stored in a large database called a Picture Archiving and Communication System (“PACS”).

A physician using a DICOM viewer may have the ability to use many tools in order to examine an image, including, but not limited to, zooming, cropping, brightening, contrasting, calculating area measurements, and performing image enhancement.

### SUMMARY

Embodiments of the present invention disclose a method, computer system, and a computer program product for determining interruptibility. The present invention may include gathering data about a task performed by a user. The present invention may include training a machine learning model based on the gathered data. The present invention may include determining a task estimate based on the trained machine learning model. The present invention may include tracking a task performance of the user in real time. The present invention may include determining an interruptibility of the user. The present invention may include providing the interruptibility of the user.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for determining interruptibility according to at least one embodiment;

FIG. 3 is a block diagram of the user dashboard according to at least one embodiment;

FIG. 4 is a block diagram of internal and external components of computers and servers depicted in FIG. 1 according to at least one embodiment;

FIG. 5 is a block diagram of an illustrative cloud computing environment including the computer system depicted in FIG. 1, in accordance with an embodiment of the present disclosure; and

FIG. 6 is a block diagram of functional layers of the illustrative cloud computing environment of FIG. 5, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for determining interruptibility. As such, the present embodiment has the capacity to improve the technical field of progress tracking systems by tracking the progress of a user performing a task, determining the user's current activity based on the tracked progress, and determining whether the user is interruptible. More specifically, the present invention may include gathering data about a task. The present invention may include training a machine learning model based on the gathered data. The present invention may include determining an initial task estimate. The present invention may include tracking a user's task performance. The present invention may include determining an interruptibility of the user. The present invention may include providing an indication of current interruptibility of the user.

As previously described, imaging may play an important role in modern medicine. Imaging techniques, including, but not limited to, X-rays, ultrasounds, CT scans, and MRIs, may show structures inside a patient's body in detail. Digital Imaging and Communications in Medicine (“DICOM”) may be a standard format that enables medical professionals to view, store, and share medical images irrespective of their geographic location or the devices they use, as long as those devices support the DICOM format. The images, along with the corresponding patient data, may be stored in a large database called a Picture Archiving and Communication System (“PACS”).

A physician using a DICOM viewer may have the ability to use many tools in order to examine an image, including, but not limited to, zooming, cropping, brightening, contrasting, calculating area measurements, and performing image enhancement.

There may be particular instances during a physician's examination of an image that the physician requires immense focus. However, physicians are often interrupted throughout their examination (i.e., read). This interruption may reduce a physician's productivity time, which may result in the physician reading fewer cases and thereby not getting through the physician's workload. The physician may also have to spend time, after an interruption, to evoke what they were doing prior to the interruption. This recollection not only adds more time to the overall read but may reduce the accuracy of the read and heighten the probability of errors. The interruptions of the physician may occur both in person and/or through telecommunication (e.g., phone calls, emails, text messages, facetimes, video calls, webex, instant messenger).

Therefore, it may be advantageous to, among other things, determine an initial time estimate (i.e., a time estimate) for a task being performed by a user, track the task performance of the user, and determine the interruptibility of the user, given the user's activity, institutional protocol, and preferences of the user.

According to at least one embodiment, the present invention may improve a user's effectiveness (e.g., productivity, accuracy) by determining the interruptibility of the user, given the user's activity, institutional protocol, and/or preferences of the user.

According to at least one embodiment, the present invention may gather data about a task and may determine data most relevant to the task a user is about to perform.

According to at least one embodiment, the present invention may include a trained machine learning model which utilizes gathered data in determining an initial time estimate for the task a user is about to perform. The machine learning model may also track in real time the task performance by the user.

The present invention may utilize click actions for tracking in real time the task performance by the user. Click actions may include, but are not limited to, cursor tracking, utilizing tools (e.g., zooming in on an image), locating prior studies for a patient that may be relevant, locating a patient's medical history and/or the preparation of a medical report for the patient, clicking through image slides (e.g., going through MRI slides), scrolling through a patient's medical history, dictation, report interaction, generating a report.

According to at least one embodiment, the present invention may provide an indication of current interruptibility of the user.

The current interruptibility of the user may be provided on a user dashboard. The user dashboard may show the time estimate for performance of the task by the user. The user dashboard may show the activity within the task being currently performed by the user. The user dashboard may show the interruptibility of the user.

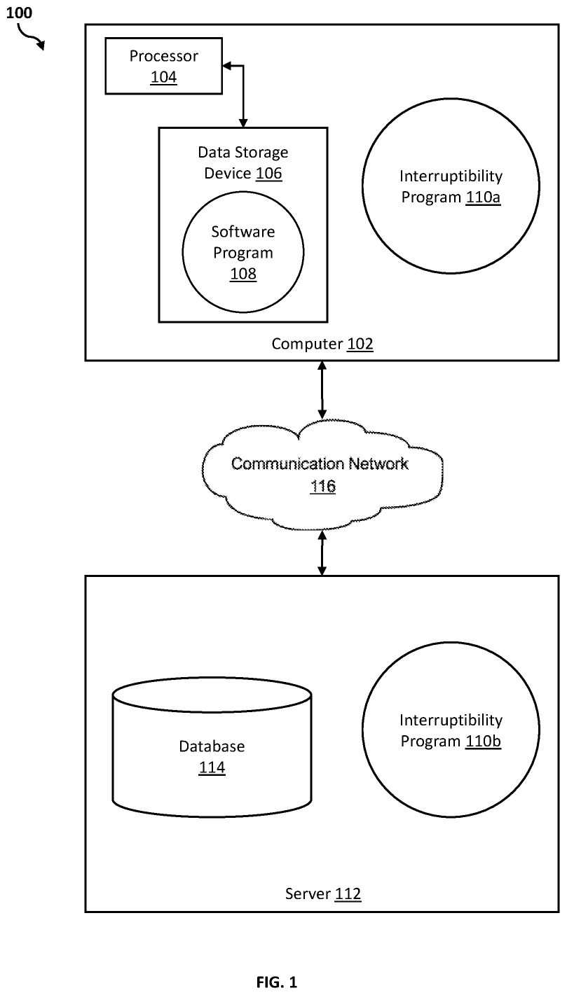

Referring to FIG. 1, an exemplary networked computer environment 100 in accordance with one embodiment is depicted. The networked computer environment 100 may include a computer 102 with a processor 104 and a data storage device 106 that is enabled to run a software program 108 and an interruptibility program 110a. The networked computer environment 100 may also include a server 112 that is enabled to run an interruptibility program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

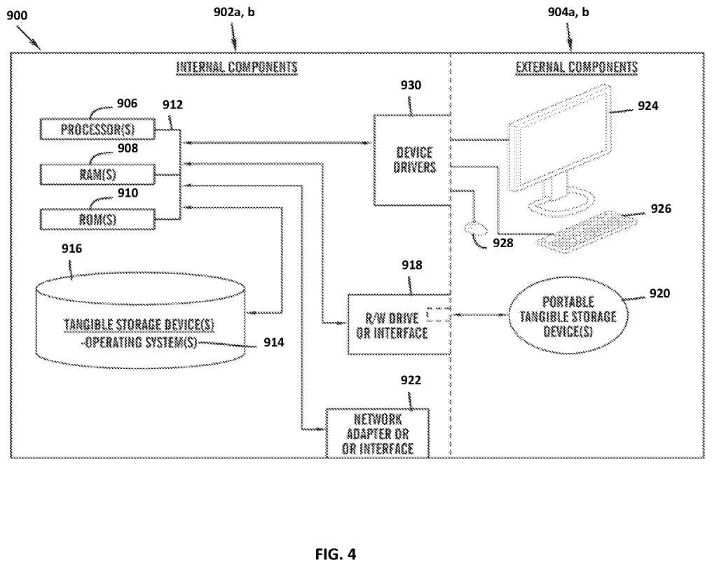

The client computer 102 may communicate with the server computer 112 via the communications network 116. The communications network 116 may include connections, such as wire, wireless communication links, or fiber optic cables. As will be discussed with reference to FIG. 4, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the interruptibility program 110a, 110b may interact with a database 114 that may be embedded in various storage devices, such as, but not limited to a computer/mobile device 102, a networked server 112, or a cloud storage service.

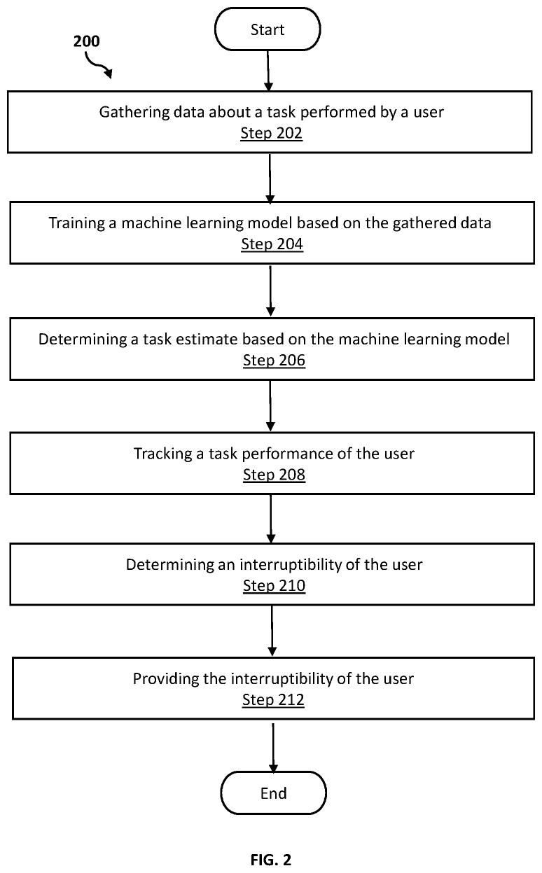

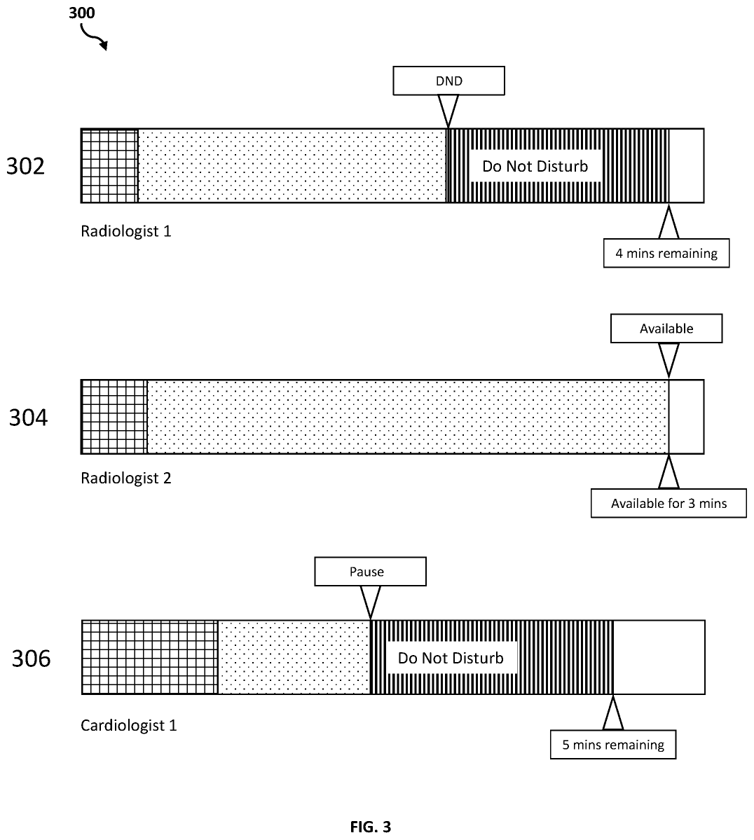

According to the present embodiment, a user using a client computer 102 or a server computer 112 may use the interruptibility program 110a, 110b (respectively) to determine task estimates, update task estimates utilizing real time tracking, provide an indication of interruptibility depending on the user's activity, institutional protocol, and preferences of the user. The determining interruptibility method is explained in more detail below with respect to FIGS. 2 and 3.

Referring now to FIG. 2, an operational flowchart illustrating the exemplary determining interruptibility process 200 used by the interruptibility program 110a and 110b (hereinafter referred to as interruptibility program 110) according to at least one embodiment is depicted.

At 202, the interruptibility program 110 may gather data about a task performed by a user.

Data about the task (e.g., reading MRI scans, reading CT scans) a user is about to perform may include, but is not limited to including, an activity within a task (e.g., the generating of a report, and/or the reviewing of films), a series of actions typically taken by a user performing the task, the modality, any characteristics of a patient, an experience level of the user performing the task, a body part and/or area of the body being examined and/or reviewed, and/or a length of time in which a task of this kind typically requires.

The interruptibility program 110 may parse through the data gathered about a task and may determine data most relevant to the task a user is performing (e.g., data specific to a patient, data specific to a task, data specific to the time it has taken for the user to perform a similar task in the past, data specific to the time it has taken other users to perform similar tasks for the same and/or a similar patient in the past). Parsing through data most relevant to the task a user is performing may allow for the gathering of more accurate data which may be used to train a machine learning model.

The interruptibility program 110 may utilize the parsed data to gather similar data from a connected database (e.g., database 114) (as will be described in more detail below).

The interruptibility program 110 may gather data about a task that a user is performing (e.g., the reviewing of films and/or the generating of a report by a radiologist, or the performing of a medical exam by a physician, among other things). Data about a task the user is performing may include, but is not limited to including, a type of task (e.g., the performing of an exam, the generating of a report, and/or the reviewing of films), a series of actions typically taken by a user performing the task, the modality, any characteristics of a patient, an experience level of the user performing the task, a body part and/or area of the body being examined and/or reviewed, and/or a length of time in which a task of this kind typically requires.

During the review of a patient's records and the generation of a report relating to same, the interruptibility program 110 may begin by gathering data from a connected database (e.g., database 114). The connected database (e.g., database 114) may, for example, be a Picture Archiving and Communication System (“PACS”) database, which may store data about a task a user (e.g., a physician) is performing and data about tasks the user has previously performed (e.g., length of time it has taken the user to perform similar tasks, length of time a radiologist has taken to prepare a report after the performance of an exam). PACS may be a medical imaging technology which provides economical storage and convenient access to images from multiple modalities (e.g., source machine types).

Electronic images and reports may be transmitted digitally via PACS. The universal format for PACS image storage and transfer may be a standard format for images. The universal format for PACS image storage and transfer may be a standard format for images used by a physician. The universal format for PACS image storage and transfer may be a standard format for images used by a radiologist. The universal format for PACS image storage and transfer may be Digital Imaging and Communications in Medicine (“DICOM”). DICOM may be a standard format for medical images. DICOM files may contain the medical images along with details about a patient.

DICOM files may be accessed using a software program (e.g., software program 108). The software program (e.g., software program 108) may be a software program used by a physician. The software program (e.g., software program 108) may be a software program used by a radiologist. The software program (e.g., software program 108) may be a DICOM viewer. DICOM files may be accessed and viewed using the DICOM viewer.

The user (e.g., the physician) may use a DICOM viewer to perform a review of the patient's records. The DICOM viewer may provide tools that enable a user to perform the review of the patient's records. DICOM viewer tools may include, but are not limited to, zooming, cropping, brightening, contrasting, calculating area measurements, image enhancement, comparing images, switching between images, and/or generating reports. A user's progress in reviewing the patient's records and/or generating a report may be tracked utilizing a user's click actions in a DICOM viewer. Click actions may include, but are not limited to, cursor tracking, utilizing tools, locating prior studies for a patient that may be relevant during the performance of the user's current task, clicking through image slides, scrolling through a patient's medical history, enabling a dictation feature, report interaction, and/or generating a report.

For the review of a given type of record (e.g., an MRI or a CT scan, among other things) the user may be expected to perform a series of actions within a DICOM viewer. User actions within a DICOM viewer may have an expected duration. The expected duration of user actions within a DICOM viewer may be based on similar previous tasks performed by the user and/or similar previous tasks performed by a different user (e.g., a second physician within the same specialty as the physician).

For example, a radiologist utilizes a DICOM viewer to perform a review of a patient's MRI scans. Upon the radiologist's upload of a first MRI scan to the DICOM viewer, the radiologist's bibliographic data (e.g., name, specialty) as well as bibliographic data of the patient, are parsed from the uploaded first MRI scan. The parsed data is then used to gather other, similar data from a connected PACS database.

For example, based on one or more user actions (e.g., the uploading of a set of MRI scans to a DICOM viewer) the interruptibility program 110 may gather data about the preparation of an MRI report. The interruptibility program 110 may have parsed through the uploaded set of MRI scans to determine that an MRI report may be generated (e.g., the task is generating an MRI report). The interruptibility program 110 may then gather data relating to the report a user is about to prepare. In an instance, the MRI report contains 20 scans, the patient has suffered a traumatic brain injury, and the radiologist has worked with similar length exams and injury histories. The interruptibility program 110 may also gather data relating to how long it has taken the radiologist to prepare similar reports in the past, what steps this radiologist takes in preparing an MRI report where the patient has suffered a traumatic brain injury, and how long it has taken other radiologists to prepare a report for this patient.

At 204, the interruptibility program 110 may train a machine learning model based on the gathered data. The machine learning model may be a time series deep learning model. The time series deep learning model may utilize gathered data in identifying task duration patterns. The time series deep learning model may receive data based on a task being performed by the user in real time. The time series deep learning model may utilize gathered data in identifying task duration patterns for similar tasks. Task duration patterns may be further categorized according to, for example, modality, patient characteristics, body part, number of images being examined.

The time series deep learning model may learn and track a user's task performance depending on the time of day. For example, a physician's read may be quicker in the morning than the afternoon.

The time series deep learning model may learn and track a user's task performance depending on the day of the week.

The time series deep learning model may learn and track from a user's task performance when the user's task performance was expected. The time series deep learning model may learn and track from a user's task performance when the user's task performance was unexpected (e.g., abnormal, task performance was longer than expected). For example, a reading physician's read may be unexpected if the task deviates from identified task duration patterns due to findings (e.g., lesion, nodule).

The time series deep learning model may be a time sequence prediction model (e.g., RNN (Recurrent Neural Network), LSTM (Long short-term memory), CNN (Convolutional Neural Network), TCN (Temporal Convolutional Networks), ED-TCN (Encoder-Decoder Temporal Convolutional Network)).

For example, the interruptibility program 110 may train the time sequence prediction model based on the gathered data. The time sequence prediction model may be an RNN. The interruptibility program may train the RNN based on the gathered data. The RNN may identify task duration patterns. The RNN may identify a user's expected click actions in a DICOM viewer for a task.

An RNN is a type of neural network that may be well-suited to time series data. RNN's may perform the same task for every element of a sequence, with the output being dependent on previous computations. LSTM is an artificial RNN architecture used in the field of deep learning. LSTM networks may classify, process, and make predictions based on time series data.

Continuing with the above example, the interruptibility program 110 may train a machine learning model based on the physician-specific and patient-specific data, among other data, gathered at 202 above.

At 206, the interruptibility program 110 may determine a task estimate based on the trained machine learning model. The interruptibility program 110 may determine an initial time estimate (i.e., a time estimate) for the task being performed by the user. For example, the interruptibility program 110 may determine that a task for a patient with a complicated health history and 20 MRI images may take longer than a task for a healthy patient and 5 MRI images.

The task being performed by the user may be comprised of interruptible and non-interruptible activities. A non-interruptible activity (e.g., reading an image, an activity determined to be non-interruptible according to institutional protocol, an activity determined to be a non-interruptible activity by the user, the read of an MRI scan, the read of a CT scan, preparation of a report, zooming in the DICOM viewer, performing a read for a patient with a complicated health history, an activity with high similarity to a non-interruptible activity, reviewing a patient's medical history) may be an activity or time within the task being performed by the user that more focus is required. An interruptible activity (e.g., logging in to a software program, entering user information, entering patient information, an activity determined to be interruptible according to institutional protocol, an activity determined to be an interruptible activity by the user, activities prior to the user opening up a CT or MRI scan) may be an activity or time within the task being performed by the user that less focus is required and/or wherein the user may be able to regain focus with ease.

The interruptibility program 110 may utilize identified task duration patterns in determining an initial time estimate (i.e., the time estimate) for a task being performed by the user. The interruptibility program 110 may determine time estimates between a user's expected click actions in determining an initial time estimate for a task performed by the user. Expected click actions may include, but are not limited to, expected cursor movements, expected tool utilization (e.g., using the zoom took within the software program), expected retrieval of patient records, expected clicking through image slides. Expected click actions may have corresponding activities.

The interruptibility program 110 may, for example, determine that based on an initial time estimate for a task being performed by the user, the user may perform a click action to generate a report 15 minutes into the task.

The interruptibility program 110 may determine that based on the initial time estimate for the task being performed by the user the future interruptibility (e.g., time remaining until the user is interruptible, interruptible time remaining for the user, future activities of the user) of the user.

At 208, the interruptibility program 110 may track the task performance of the user. The interruptibility program 110 may track the task performance of the user in real time. The interruptibility program 110 may track the task performance of the user in a software program (e.g., software program 108). The software program (e.g., software program 108) may be a software program used by a physician. The software program (e.g., software program 108) may be a software program used by a radiologist. The software program (e.g., software program 108) may be a DICOM viewer. The interruptibility program may track the task performance of the user in the DICOM viewer. The interruptibility program 110 may track click actions of the user in the software program (e.g., software program 108).

The interruptibility program 110 may track the click actions of the user in the software program (e.g., software program 108). The interruptibility program 110 may track the click actions of the user in the DICOM viewer to determine the task progress of the user.

Click actions may include, but are not limited to, cursor tracking, utilizing tools (e.g., zooming in on an image), locating prior studies for a patient that may be relevant during a review of the patient's medical history and/or the preparation of a medical report for the patient, clicking through image slides, scrolling through a patient's medical history, dictation, report interaction, and generating a report.

The interruptibility program 110 may determine the activity being performed by the user based on click actions. The activity being performed by the user may be an interruptible activity or a non-interruptible activity. For example, if the DICOM viewer zoom tool is being used, then the interruptibility program 110 may determine that the user is reading an image. Reading an image may be a non-interruptible activity.

The machine learning model may update the initial time estimate for performance of a task by the user based on actions taken by the user in the software program (e.g., software program 108). For example, if an initial time estimate for a task being performed by the user is 20 minutes, with an estimate of a click action to generate a report at 15 minutes, and the user generates a report at 17 minutes, the machine learning model may update the time estimate for performance of the task to 22 minutes.

Continuing with the above example, physician-specific data may be a series of click actions the physician has performed for similar tasks (e.g., opening the DICOM viewer, accessing a database, entering patient information, entering user information, opening MRI scans, going through the MRI scans, dictation, zooming in on MRI scans, enhancing an image area).

The interruptibility program 110 may utilize gathered data in identifying task duration patterns for a particular user. The interruptibility program 110 may utilize gathered data in identifying task duration patterns for a type of task, modality, patient medical history.

The interruptibility program 110 may utilize the identified task duration patterns for the particular user to determine future interruptibility (e.g., time remaining until the user is interruptible, interruptible time remaining for the user, future activities of the user) of the particular user.

The interruptibility program 110 may feed data gathered while tracking the task performance of the user back into the machine learning model. The interruptibility program 110 may feed data gathered while tracking the task performance of the user to the machine learning model in real time. The machine learning model may be a time series deep learning model. The time series deep learning model may be an RNN.

At 210, the interruptibility program 110 may determine the interruptibility of the user. The interruptibility program 110 may determine the interruptibility of the user based on the user's expected click actions in the software program (e.g., software program 108) for a task. The interruptibility program may determine the future interruptibility (e.g., time remaining until the user is interruptible, interruptible time remaining for the user) of the user. Click actions may have an associated activity. An activity may be an interruptible activity or a non-interruptible activity. Interruptibility may be determined based on the attentiveness required for the user activity.

For example, the interruptibility program 110 may determine that the combination of using the zoom tool and brightness tool within the software program (e.g., software program 108) is associated with the activity of reading an MRI image. The interruptibility program 110 may determine that the reading of the MRI image using the zoom tool and brightness tool requires a level of attentiveness from the user. The interruptibility program 110 may determine the user is performing a non-interruptible activity based off the attentiveness required for those click actions.

The interruptibility program 110 may determine the interruptibility of the user based on the user's expected click actions in the DICOM viewer for a task. The interruptibility program 110 may determine the interruptibility of the user based on the user's activity associated with click actions in a DICOM viewer.

The interruptibility program 110 may determine the interruptibility of the user by utilizing gathered data of a similar task. The interruptibility program 110 may determine the interruptibility of the user by determining the similarity (e.g., percentage of how similar a task may be, percentage of how similar an activity may be) between the task a user is performing and a task the user has previously performed.

For example, the user may be performing a read of an MRI scan for a patient. The user may have previously performed a read of a CT scan for the same patient. The interruptibility program 110 may determine the task previously performed (e.g., read of the CT scan) has a high similarity (e.g., 90%) to the task the user is performing (e.g., read of an MRI scan). The interruptibility program 110 may determine similar activities within the similar tasks have corresponding interruptibility. Continuing with the above example, entering patient information during the performance of a read of an MRI scan may be an interruptible activity. Accordingly, entering patient information during the performance of a read of a CT scan may be an interruptible activity.

Non-interruptibility of the user may be based on the user's activity. Non-interruptibility may be determined based on the attentiveness required for the user activity. For example, if a radiologist is using DICOM viewer tools with respect to an MRI scan and dictating a report this may be a non-interruptible activity.

Non-interruptible activities may also be determined by institutional protocol. For example, an institution (e.g., hospital, doctor's office, urgent care center) may determine that once a user begins preparing a report the user is non-interruptible. Here, the interruptibility program 110 may track the user's click actions to determine when the user has begun preparing a report.

Non-interruptible activities may also be determined by user preference. For example, the user may determine that once the user has opened MRI scans the user is non-interruptible until a report is generated. The interruptibility program 110 may track the user's click actions to determine when the user has opened the MRI scans.

The interruptibility program 110 may also allow for exceptions to non-interruptible activities. Exceptions to non-interruptible activities may be determined by user preference. For example, the user may be non-interruptible but allow interruptions from their significant other or family members.

The interruptibility program 110 may also determine degrees of interruptibility. The interruptibility program 110 may determine certain activities within a task are non-interruptible while other activities within a task are recommended non-interruptible. For example, an interrupting technician or physician may determine that even though an activity is recommended non-interruptible this interruption is rising to an interruptible threshold.

At 212, the interruptibility program 110 may provide the interruptibility of the user. The interruptibility program 110 may provide current interruptibility (i.e., the interruptibility) on a user dashboard. The interruptibility program may provide future interruptibility (e.g., time remaining until the user is interruptible, interruptible time remaining for the user, future activities of the user, future tasks of the user, time series of future interruptibility) on the user dashboard. The user dashboard may be comprised of one or more progress status indicators. The progress status indicators may provide progress information for one or more users. Progress information may include, but is not limited to, whether the user is interruptible or non-interruptible, “Do Not Disturb” as an indication of the current interruptibility of a user, time remaining until a user is interruptible, interruptible time remaining for a user, the current activity of a user.

The interruptibility program 110 may provide current interruptibility of the user on a user dashboard. The interruptibility program 110 may provide the future interruptibility (e.g., time remaining until the user is interruptible, interruptible time remaining for the user, future activities of the user, future tasks of the user, time series of future interruptibility) of the user. The user dashboard may include one or more progress status indicators for one or more users. The progress status indicator may show a time estimate for performance of a task for the user, the current activity of the user, and the interruptibility of the user. The time estimate for performance of a task by the user may be in the form of a countdown timer (e.g., hours, minutes and seconds).

As an alternate embodiment, for example, the interruptibility program 110 may pair with a calendar of an institution (e.g., hospital, doctor's office, urgent care center) which might have appointments (e.g., tasks) for patients listed.

For example, a user may want to block off time as non-interruptible to prepare a report. The interruptibility program 110 may analyze the user's calendar and determine that based off the type of report being generated by the user, it may take the user up to 30 minutes to prepare the report, and further based on the fact that the user has 3 appointments (e.g., tasks) in the hours that will follow the report preparation, the interruptibility program 110 may determine now is not an optimal time to prepare a report.

The interruptibility program 110 may utilize the determined future interruptibility in determining appointment scheduling. For example, the interruptibility program 110 may recommend to a particular radiologist to a referring physician based on the radiologist's determined future interruptibility.

The interruptibility program 110 may allow the user to be interrupted during the preparation of the report.

Referring now to FIG. 3, a block diagram of the user dashboard 300 used by the interruptibility program 110 according to at least one embodiment is depicted. At 302 a progress status indicator for a Radiologist 1 is depicted. At 304 a progress status indicator for a Radiologist 2 is depicted. At 306 a progress status indicator for a Cardiologist 1 is depicted.

At 302 the progress status indicator for Radiologist 1 shows that Radiologist 1 is non-interruptible which is indicated by the “Do Not Disturb” status. At 302 the progress status indicator depicts 4 minutes remaining before Radiologist 1 will be interruptible. The boxed pattern depicts an interruptible time in the beginning of the task being performed by Radiologist 1. The dotted pattern depicts Radiologist 1's completed progress within non-interruptible activities. The vertical line pattern depicts the remaining non-interruptible time. The clear portion at the far right of the progress status indicator depicts the amount of interruptible time Radiologist 1 will have at the end of the task.

At 304 the progress status indicator for Radiologist 2 shows that Radiologist 2 is interruptible which is indicated by the “available” status. At 304 the progress status indicator depicts 3 minutes remaining before Radiologist 2 will be non-interruptible. The boxed pattern depicts an interruptible time in the beginning of the task being performed by Radiologist 2. The dotted pattern depicts Radiologist 2's completed progress within non-interruptible activities. The clear portion at the far right of the progress status indicator represents the 3 minutes remaining interruptible time Radiologist 2 has remaining for this task.

At 306 the progress status indicator for Cardiologist 1 shows that Cardiologist 1 is currently paused in the performance of the task. At 306 the progress status indicator depicts 5 minutes remaining from the time the task is resumed before Cardiologist 1 will be interruptible. The boxed pattern depicts an interruptible time in the beginning of the task being performed by Cardiologist 1. The dotted pattern depicts Cardiologist 1's completed progress within non-interruptible activities. The vertical line pattern depicts the remaining non-interruptible time. The clear portion at the far right of the progress status indicator depicts the amount of interruptible time Cardiologist 1 will have at the end of the task.

For example, a person (e.g., a technician, referring physician) wanting to interrupt the user may utilize the user dashboard 300 to determine there is a better time or a more convenient user to interrupt.

It may be appreciated that FIGS. 2 and 3 provide only an illustration of one embodiment and do not imply any limitations with regard to how different embodiments may be implemented. Many modifications to the depicted embodiment(s) may be made based on design and implementation requirements.

FIG. 4 is a block diagram 900 of internal and external components of computers depicted in FIG. 1 in accordance with an illustrative embodiment of the present invention. It should be appreciated that FIG. 4 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902a, b and external components 904a, b illustrated in FIG. 4. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the interruptibility program 110a in client computer 102, and the interruptibility program 110b in network server 112, may be stored on one or more computer-readable tangible storage devices 916 for execution by one or more processors 906 via one or more RAMs 908 (which typically include cache memory). In the embodiment illustrated in FIG. 4, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the interruptibility program 110a and 110b can be stored on one or more of the respective portable computer-readable tangible storage devices 920, read via the respective R/W drive or interface 918 and loaded into the respective hard drive 916.

Each set of internal components 902a, b may also include network adapters (or switch port cards) or interfaces 922 such as a TCP/IP adapter cards, wireless wi-fi interface cards, or 3G or 4G wireless interface cards or other wired or wireless communication links. The software program 108 and the interruptibility program 110a in client computer 102 and the interruptibility program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the interruptibility program 110a in client computer 102 and the interruptibility program 110b in network server computer 112 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

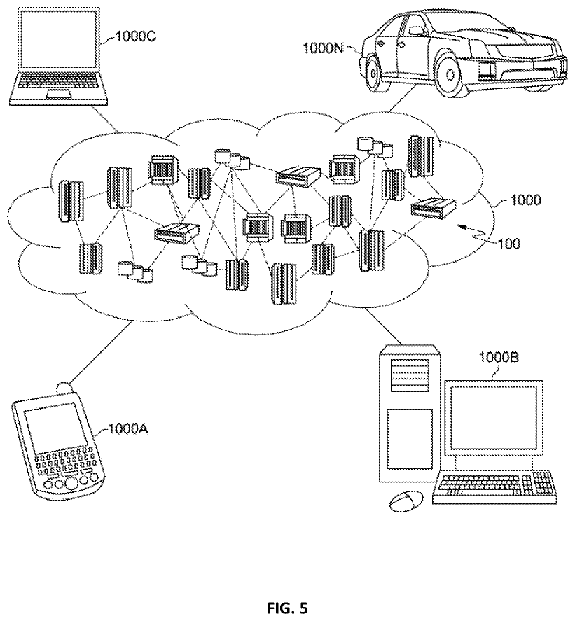

Referring now to FIG. 5, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 5 are intended to be illustrative only and that computing nodes 100 and cloud computing environment 1000 can communicate with any type of computerized device over any type of network and/or network addressable connection (e.g., using a web browser).

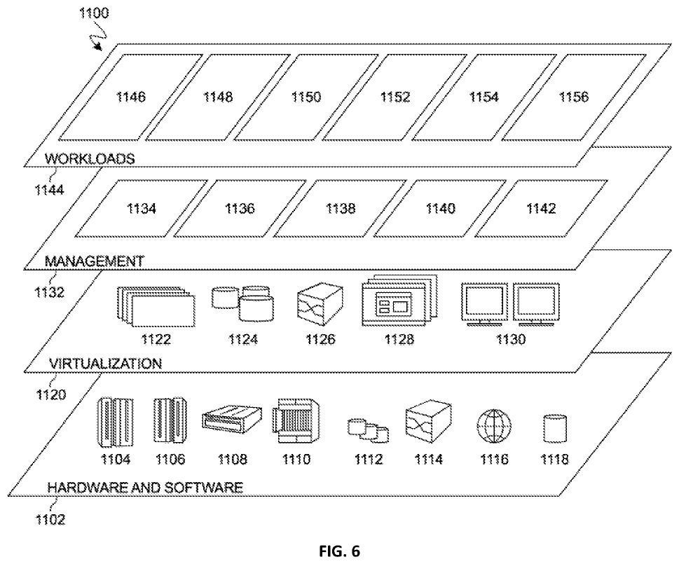

Referring now to FIG. 6, a set of functional abstraction layers 1100 provided by cloud computing environment 1000 is shown. It should be understood in advance that the components, layers, and functions shown in FIG. 6 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and determining interruptibility 1156. An interruptibility program 110a, 110b provides a way to determine a time estimate for the task being performed by the user, track the performance of the user in real time, and determine the interruptibility of the user.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for determining interruptibility, the method comprising:
gathering data about a task performed by a user;
training a machine learning model based on the gathered data, wherein the machine learning model is a time series deep learning model;
determining a task estimate based on the trained machine learning model, wherein the task estimate is a time estimate for the task being performed by the user;
tracking a task performance of the user in real time, wherein the task performed by the user is within a software program;
determining an interruptibility of the user; and
providing the interruptibility of the user.

2. The method of claim 1, wherein the time series deep learning model is a time sequence prediction model, and wherein the time series prediction model receives data based on tracking the task performance of the user in real time.

3. The method of claim 1, wherein determining the time estimate for the task performed by the user further comprises:
analyzing one or more expected click actions.

4. The method of claim 1, wherein tracking the task performance within the software program of the user in real time further comprises:
using a DICOM viewer as the software program; and
tracking one or more click actions, wherein the one or more click actions are performed within the DICOM viewer, each of the one or more click actions having a corresponding activity, and wherein the corresponding activity is either a non-interruptible activity or an interruptible activity.

5. The method of claim 1, wherein tracking the task performance within the software program of the user in real time further comprises:
tracking one or more click actions within the software program of the user; and
updating the time estimate for the task being performed by the user.

6. The method of claim 1, wherein determining the interruptibility of the user further comprises:
identifying one or more similar tasks previously performed by the user; and
determining a similarity of the task performed by the user and the one or more similar tasks previously performed by the user.

7. The method of claim 1, wherein providing an indication of the current interruptibility of the user further comprises:
utilizing a user dashboard, wherein the user dashboard is comprised of one or more progress status indicators, and wherein the progress status indicators provide a plurality of progress information for one or more users.

8. A computer system for determining interruptibility, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
gathering data about a task performed by a user;
training a machine learning model based on the gathered data, wherein the machine learning model is a time series deep learning model;
determining a task estimate based on the trained machine learning model, wherein the task estimate is a time estimate for the task being performed by the user;
tracking a task performance of the user in real time, wherein the task performed by the user is within a software program;
determining an interruptibility of the user; and
providing the interruptibility of the user.

9. The computer system of claim 8, wherein the time series deep learning model is a time sequence prediction model, and wherein the time series prediction model receives data based on tracking the task performance of the user in real time.

10. The computer system of claim 8, wherein determining the time estimate for the task performed by the user further comprises:
analyzing one or more expected click actions.

11. The computer system of claim 8, wherein tracking the task performance within the software program of the user in real time further comprises:
using a DICOM viewer as the software program; and
tracking one or more click actions, wherein the one or more click actions are performed within the DICOM viewer, each of the one or more click actions having a corresponding activity, and wherein the corresponding activity is either a non-interruptible activity or an interruptible activity.

12. The computer system of claim 8, wherein tracking the task performance within the software program of the user in real time further comprises:
tracking one or more click actions within the software program of the user; and
updating the time estimate for the task being performed by the user.

13. The computer system of claim 8, wherein determining the interruptibility of the user further comprises:
identifying one or more similar tasks previously performed by the user; and
determining a similarity of the task performed by the user and the one or more similar tasks previously performed by the user.

14. The computer system of claim 8, wherein providing an indication of the current interruptibility of the user further comprises:
utilizing a user dashboard, wherein the user dashboard is comprised of one or more progress status indicators, and wherein the progress status indicators provide a plurality of progress information for one or more users.

15. A computer program product for determining interruptibility, comprising:
one or more non-transitory computer-readable storage media and program instructions stored on at least one of the one or more tangible storage media, the program instructions executable by a processor to cause the processor to perform a method comprising:
gathering data about a task performed by a user;
training a machine learning model based on the gathered data, wherein the machine learning model is a time series deep learning model;
determining a task estimate based on the trained machine learning model, wherein the task estimate is a time estimate for the task being performed by the user;
tracking a task performance of the user in real time, wherein the task performed by the user is within a software program;
determining an interruptibility of the user; and
providing the interruptibility of the user.

16. The computer program product of claim 15, wherein determining the time estimate for the task performed by the user further comprises:
analyzing one or more expected click actions.

17. The computer program product of claim 15, wherein tracking the task performance within the software program of the user in real time further comprises:
using a DICOM viewer as the software program; and
tracking one or more click actions, wherein the one or more click actions are performed within the DICOM viewer, each of the one or more click actions having a corresponding activity, and wherein the corresponding activity is either a non-interruptible activity or an interruptible activity.

18. The computer program product of claim 15, wherein tracking the task performance within the software program of the user in real time further comprises:
tracking one or more click actions within the software program of the user; and
updating the time estimate for the task being performed by the user.

19. The computer program product of claim 15, wherein determining the interruptibility of the user further comprises:
identifying one or more similar tasks previously performed by the user; and
determining a similarity of the task performed by the user and the one or more similar tasks previously performed by the user.

20. The computer program product of claim 15, wherein providing an indication of the current interruptibility of the user further comprises:
utilizing a user dashboard, wherein the user dashboard is comprised of one or more progress status indicators, and wherein the progress status indicators provide a plurality of progress information for one or more users.
